SEQUENCE LISTING (13 sequences)

SEQ ID NO: 1 - DNA, 9876 bp, Artificial
  Nucleotide sequence of V5 embodiment (delta rt SHIV KU-2)
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaagaatggaccaaaattgaagcagtggccattatcaaaagaaaagatagttgcattaagagaaatctgggaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggattagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcggcactcgtggagatgggggtggagatggggcaccatgctacttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagacaatatgggtggagctatttccatgaggcggtccaggcagtctagagatctgcgacagagactcttgcgggcgcgtggggagacttatgggagactcttagaagaggtggaagatggatactcgcgatccccaggaggattagacaagggcttgagctcactctcttgtgagggacagaaatacaatcagggacagtatatgaatactccatggagagacccagctgaagagagagaaaaattagcatacagaaaacaaaatatggatgatatagatgaggaagatgataacttggtaggggtatcagtgaggccaagagttcccctaagaacaatgagttacaaattggcaatagacatgtctcattttataaaagaaaagggggaactggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 2 - DNA, 762 bp, Artificial
  Nucleotide sequence of deleted portion of rt gene in v5 (delta rt SHIV KU-2)
agccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggatacc

SEQ ID NO: 3 - DNA, 9984 bp, Artificial
  Nucleotide sequence of v6 embodiment (delta rt delta 3′ LTR SHIV KU-2)
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaagaatggaccaaaattgaagcagtggccattatcaaaagaaaagatagttgcattaagagaaatctgggaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggattagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcggcactcgtggagatgggggtggagatggggcaccatgctacttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagattcgagatgggtggagctatttccatgaggcggtccaggcagtctagagatctgcgacagagactcttgcgggcgcgtggggagacttatgggagactcttagaagaggtggaagatggatactcgcgatccccaggaggattagacaagggcttgagctcactctcttgtgagggacagaaatacaatcagggacagtatatgaatactccatggagagacccagctgaagagagagaaaaattagcatacagaaaacaaaatatggatgatatagatgaggaagatgataacttggtaggggtatcagtgaggccaagagttcccctaagaacaatgagttacaaattggcaatagacatgtctcattttataaaagaaaagggggaactggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgagcggccgcctgcaggtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaat

SEQ ID NO: 4 - DNA, 411 bp, Artificial
  Nucleotide sequence of portion of 3-prime LTR deleted in v6 embodiment (delta rt delta 3′ LTR SHIV KU-2)
aacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 5 - DNA, 9715 bp, Artificial
  Nucleotide sequence of v7 embodiment (delta rt delta 3′ LTR SHIV KU-2)
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcccgaacagggacgcgaaagcgaaagtagaaccagaggagctctctcgacgcaggactcggcttgctgaagcgcgcacagcaagaggcgaggggcggcgactggtgagtacgccaatttttgactagcggaggctagaaggagagagagatgggtgcgagagcgtcggtattaagcgggggagaattagataaatgggaaaaaattcggttaaggccagggggaaagaaaaaatataagttaaaacatatagtatgggcaagcagggagctagaacgattcgcagtcaatcctggcctgttagaaacatcagaaggctgcagacaaatattgggacagctacagccatcccttcagacaggatcagaagaacttagatcattatataatacagtagcaaccctctattgtgtacatcaaaggatagatgtaaaagacaccaaggaagctttagagaagatagaggaagagcaaaacaaaagtaagaaaaaggcacagcaagcagcagctgcagctggcacaggaaacagcagccaggtcagccaaaattaccctatagtgcagaacctacaggggcaaatggtacatcaggccatatcacctagaactttaaatgcatgggtaaaagtagtagaagaaaaggctttcagcccagaagtaatacccatgttttcagcattatcagaaggagccaccccacaagatttaaacaccatgctaaacacagtggggggacatcaagcagccatgcaaatgttaaaagagactatcaatgaggaagctgcagaatgggatagagtgcatccagtgcatgcagggcctattgcaccaggccaaatgagagaaccaaggggaagtgacatagcaggaactactagtacccttcaggaacaaataggatggatgacaaataatccacctatcccagtaggagaaatctataaaagatggataatcctgggattaaataaaatagtaagaatgtatagccctaccagcattctggacataagacaaggaccaaaggaaccctttagagattatgtagaccggttctataaaactctaagagccgaacaagcttcacaggatgtaaaaaattggatgacagaaaccttgttggtccaaaatgcaaacccagattgtaagactattttaaaagcattgggaccagcagctacactagaagaaatgatgacagcatgtcagggagtggggggacccggccataaagcaagagttttggctgaagccatgagccaagtaacaaatccagctaacataatgatgcagagaggcaattttaggaaccaaagaaagactgttaagtgtttcaattgtggcaaagaagggcacatagccaaaaattgcagggcccctaggaaaaagggctgttggagatgtggaagggaaggacaccaaatgaaagattgcactgagagacaggctaattttttagggaagatctggccttcctacaagggaaggccagggaattttcttcagagcagaccagagccaacagccccaccagaagagagcttcaggtttggggaggagaaaacaactccctctcagaagcaggagccgatagacaaggaactgtatcctttaacttccctcagatcactctttggcaacgacccctcgtcacaataaggataggggggcaactaaaggaagctctattagatacaggagcagatgatacagtattagaagaaatgaatttgccaggaaaatggaaaccaaaaatgatagggggaattggaggttttatcaaagtaagacagtacgatcagatacctgtagaaatctgtggacataaagctataggtacagtattagtaggacctacacctgtcaacataattggaagaaatctgttgactcagattggttgtactttaaatttccccattagtcctattgaaactgtaccagtaaaattaaagccaggaatggatggcccaaaagttaagcaatggccattgacagaagaaaaaataaaagcattagtagagatatgtacagaaatggaaaaggaagggaaaatttcaaaaattgggcctgaaaatccatacaatactccagtatttgctataaagaaaaaagacagtactaaatggagaaaactagtagatttcagagaacttaataaaagaactcaagacttctgggaagttcagttaggaataccacaccccgcagggttaaaaaagaaaaaatcagtaacagtattggatgtgggtgatgcatacttttcagttcccttagataaagactttagaaagtatactgcatttaccatacctagtataaacaatgagacaccagggattagatatcagtacaatgtgctgccacagggatggaaagggtcaccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcggcactcgtggagatgggggtggagatggggcaccatgctacttgggatgttgatgatctgtagtgctacagaaaaattgtgggtcacagtctattatggggtacctgtgtggaaggaagcaaccaccactctattttgtgcatcagatgctaaagcatatgatacagaggcacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaattgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcatttcacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcatggacaatgctaaaaccataatagtacagctgaacacatctgtagaaattaattgtacaagacccagcaacaatacaataaaaagaatccgtatccagagaggaccagggagagcatttgttacaatgggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtggcacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaagggcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacatgacctggatggagtgggacagagaaattaacaattacacaagcttaatacactccttaattgaagaatcgcaaaaccagcaagaaaagaatgaacaagaattattggaattagataaatgggcaagtttgtggaattggtttgacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctacggagcctgtgcctcttcagctaccaccgcttgagagacttactcttgattgtaacgaggactgtggaacttctgggacgcagggggtgggaagccctcaaatattggtggaatctcctacagtattggagtcaggaactaaagaatagtgctgttagcttgctcaatgccatagccatagcagtagctgagggaacagatagggttatagaagtagtccaaggagcttgtagagctattcgctacatacctagaagaataagacagggcttggaaaggattttgctataagattcgacatgggtggcaagtggtcaaaacgtagtatgggtggatggtctgctataagggaaagaatgagacgagctgagccacgagctgagccagcagcagatggggtgggagcagtatctcgagacctggaaaaacatggagcaatcacaagtagcaatacagcagctactaatgctgattgtgcctggctagaagcacaagaggaggaagaggtgggttttccagtcagacctcaggtacctttaagaccaatgacttacaaggcagctttagatattagccactttttaaaagaaaaggggggactggaagggctaatttggtcccaaagaagacaagagatccttgatctgtggatctaccacacacaaggctacttccctgattggcagaattacacaccagggccagggatcagatatccactgacctttggatggtgcttcaagctagtaccagttgagccagagaaggtagaagaggccaatgaaggagagaacaacagcttgttacaccctatgagcctgcatgggatggaggacgcggagaaagaagtgttagtgtggaggtttgacagcaaactagcatttcatcacatggcccgagagctgcatccggagtactacaaagactgctgagcggccgcctgcaggtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaat

SEQ ID NO: 6 - DNA, 818 bp, Artificial
  Nucleotide sequence of portion of 3-prime LTR deleted in v7 embodiment (delta rt delta 3′ LTR SHIV KU-2)
tggaagggatcttttacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 7 - DNA, 9994 bp, Artificial
  Nucleotide sequence of delta 4-SHIV KU-2
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcccgaacagggacgcgaaagcgaaagtagaaccagaggagctctctcgacgcaggactcggcttgctgaagcgcgcacggcaagaggcgaggggcggcgactggtgagtacgccatttttgactagcggaggctagaaggagagagatgggtgcgagagcgtcaatattaagcgggggacaattagatagatgggaaaaaattcggttacggccagggggaaagaaaagatataagttaaaacatatagtatgggcaagcagagagctagaacgattcgcagttaaccctggcctgttagaaacagcagaaggctgtagacaaatactgggacagctacaaccatcccttcagacaggatcagaggaacttaaatcattatttaatacaatagctaccctctattgtgtacatcaaagaatagagataaaagacaccaaggaagctttagataagatagaggaagagcaaaacaaaagtaagaaaaaagcacagcaagcagcagctgacacaggaaacagcagcagccaagtcagccaaaattaccctatagtgcagaacgctcagggacaaatggtacatcaggccatatcacctagaactttaaatgcatgggtaaaagtagtagaagaaaaggcttttaacccagaagtaatacccatgtttgcagcattgtcagaaggagccaccccacaagatttaaacaccatgctaaacacagtggggggacatcaagcagccatgcaaatattaaaagagactatcaatgaggaagctgcagaatgggatagattgcatccagtacatgcagggcctattgcaccaggccaaatgagagaaccaaggggaagtgacatagcaggaactactagtacccttcaggaacaaataggatggatgacaaataatccacctatcccagtaggagaaatctataaaaaatggataatcatgggattaaataaaattgtaaggatgtatagccctaccagtattctggacataagacaaggaccaaaggaaccctttagagactatgtagaccggttctataaaactctaagagccgagcaagcttcacaggaagtaaaaaattggatgacagaaaccttgttggtccaaaattcaaaccccgattgtaagactattttaaaagcattaggaccaggagctacactagaagaaatgatgacagcatgccagggagtgggaggacctggccataaagcaagagttttggcagaagcaatgagccaagtaacaaatccaacggccgtgatgatgcagaaaagcaattttaggggccaaagaaaaattgttaagtgttttaattgtggcaaagaagggcacatagccaaaaattgcagggctcctagaaaaaagggctgttggaaatgtggaaaggaaggacaccaaatgaaagattgtactgaaagacaggctaattttttagggaagatctggccttcctacaagggaaggccagggaattttcctcaaagcaggctagaaccaacagccccaccagaagcgagcttcaggtttggggaggagacaacaactccccctcagaagcaggagacgatagacaaggaggtgtatcctttaacctccctcagatcactctttggcaacgacccctcgtcacaataaagataggggggcaactaaaagaagctctattagatacaggagcagatgatacagtgttagaagacatgaatttgccaggaaaatggaaaccaaaaatgatagggggaattggaggatttatcaaagtaaaacagtatgatcagatacccatagaaatctgtggacataaaactataggtacagtattaataggacctacacctgtcaacataattggaaggaatttgttgactcagcttggttgcactttaaattttcccattagtcctattgaaactgtaccagtaaaattaaagccaggaatggatggcccaaaagttaagcaatggccattgacagaagaaaaaataaaagcattaatggagatatgcacagaaatggaaaaggaagggaaaatttcaaaaattgggcctgaaaatccatacaatactccagtgtttgccataaagaaaaaagacagtactaagtggagaaaattagtagatttcagagaacttaataagaaaactcaagacttctgggaggttcaattaggaataccacatcccgcggggttaaaaaagaaaaagtcagtaacagtactggatgtgggtgatgcatacttctcagttcccttagatgaagattttaggaagtatactgcatttaccatacctagtataaacaatgagacatcaggaattagatatcagtacaatgtgcttccacagggatggaaagggtcaccatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttttggaagaactgaaagaagaagctttaaaacattttgatcctcgcttgctaactgcccttggtaatcatatctataatcgtcacggagacactctagagggagcaggagaactcattagaatcctccaacgagcgctcttcatgcatttcagaggcggatgcatccactccagaatcggccaacctgagggaggaaatcctctctcagctataccgccctctagaagcattctgtagagcaagaaatggagccagtagatcctagactagagccctggaagcatccaggaagtaagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagagatatcagaattatcagcacttgtggagagagggcatcttgctccttgggatattaatgatatgtagcactgcaggacaattgtgggtcacagtctattatggggtacctgtgtggagagaagcaaccaccactctattttgtgcatcagatgctaaagcatatgatacagaggtgcataatgtctgggccacacatgcctgtgtacccacagaccccagcccacaagaaatggcattggaaaatgtgacagaaaattttgacatgtggaaaaataatatggtagaacagatgcatgaagatataatcagcttatgggatcaaagcctaaagccttgtgtaaaattaactccactatgtgttactttaaattgcactgatgtaaagagaaatgctactagtaacactagtagtagctgggaaaggatggaaccaggagaaataaaaaactgctctttcaatgtcacctcaaatataagagataagatgcggaaagaatatgcactcttttataaacttgatgtaataccaataaataatactagtgataatagtgctaaatatagattgataagttgtaacacctcagtccttacacaagcttgtccaaaaatatcctttgagccaattccaatacattattgtaccccggctggttttgcgcttctgaagtgtaatgataaggagttcaatggaacgggaccatgtaaaaatgtcagcacagtacaatgtacacatggaatcaagccagtagtatcaactcaactgctgttaaatggcagtctatcagaaggaggggttgtaattagatctcaaaatttcacaaacaatgctaaaaccataatagtacagctgaatgaaactgtagaaattaattgtacaaggcccaacaacaatacaagaagaagtataaatataggaccagggagagcattttatgcagcagaacaaataataggagatataagacaagcacattgtaacattagtagagcaaaatggaataacactttaaaactgatagttggaaaattacaagaacaatttgggaagaaaacaataatctttaatcaatcctcaggaggagaccctgagattgtaacacacagttttaattgtggaggggaatttttctactgtgattcaacacaactgtttaacagtacttggacgaatgaaaataacgggtccaacactaaagggaatgacacaatcatactaccatgcagaataaaacaaattgtaaacctgtggcaggaagtaggaaaagcaatgtatgcccctcccatcagaagaccaattagatgctcatcaaatattacagggctgctactaacaagagatggtggtcctaataggacgaacgagacattcagacctggaggaggagatatgagggacaattggagaagtgaattatacaaatataaagtagtaaaaattgaaccattaggagtagcacccaccaaggcaaagagaagagtggtgcaaagagaaaaaagagcagtgggaataggagctctgttccttgggttcttgggaacagcaggaagcactatgggcgcagcgtcactgacgctgacggtacaggccagacaattattgtctggtatagtgcaacagcagaacaatttgctgagagctattgaagcgcaacaacatctgttgcagctcacagtctggggcatcaagcagctccaggcaagagtcctggctgtggaaagatacctaagggatcaacagctcctgggaatttggggttgctctggaaaactcatttgcaccactgctgtgccttggaacactagttggagtaataaatctctagatgacatttggaacaacatgacttggatgcagtgggaaagagaaattgacaattacacaaacacaatatacaccttacttcaggaatcacaactccaacaagaacagaatgaaaaagaactattggaattggataaatgggcaagtttgtggaattggttcgatataacaagttggctgtggtatataaaaatattcataatgatagtaggaggcttgataggtttaagaatagtttttactgtactttctatagtgaatagagttaggaagggatactcaccattatcgttccagacccaccgcccagctccagggggacccgacaggcccgaaggaatcgaagaagaaggtggagagagagacagagaaagatccaatcaattagtggatggattcttagcaattatctgggtcgacctgcggaacctgtgcctcttcagctaccaccgcttgagagacttactcttgattgcaacgaggattgtggaacttctgggacgcagggggtgggaagccctcaaatattggtggaatctcctgcagtattggagtcaggaactgaagaatagtgctgttagcttgcttaatgccatagccatagcagtagctgaggggacagatagaattatagaagtagtacaaaggggggttagagctgttcttaacatacccacaagaataagacagggagcggaaaggcttcttgtataagatgggtggcaagttgtcaaaaagtaagatgcctggatggtctactataagggaaagaatgagacgagctcagccagcagcagagccagcagcagttggggtgggagcagcatctcgagacctggaaagacatggagcactcacaagtagcaatacagcagctaacaatgctgattgtgcctggctagaagcacaagaggacgaggaagtgggttttccagtcagacctcaggtacctcttaggccaatgacttacaagggagctgtagatcttagccactttttaaaagaaaaggggggactggaagggttagtttactcccaaaaaagacaagacatccttgatctgtgggtctaccacacacaaggctacttccctgattggcagaactacacaccagggccagggatcagatatcccctgacctttggatggtgcttcaagctagtaccagttgatccagataaggtagaagaggccaatgaaggagagaacaactgcttattacaccctatggcccagcatgggatggatgacccagagaaagaagtgttagtgtggaagtttgacagccgcctagcatttcatcacatggcccgagagctgcatccggagtactacaaagactgctgagcggccgccctgcaggtcgacctcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattc

SEQ ID NO: 8 - DNA, 4981 bp, Artificial
  Nucleotide sequence of SHIV KU-2 removed in delta 4-SHIV KU-2
cgcccgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtgagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaataggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgcctatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaagaatggaccaaaattgaagcagtggccattatcaaaagaaaagatagttgcattaagagaaatctgggaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggattagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcgtgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgccatcagaaaggagaggctatacatgggcaggtaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtag

SEQ ID NO: 9 - DNA, 472 bp, Artificial
  Nucleotide sequence of SHIV KU-2 removed in delta 4-SHIV KU-2
atggaggaggaaaagaggtggatagcagttcccacatggaggataccggagaggttagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagcaccaggtaccaagcctacagtacttagcactgaaagtagtaagcg

SEQ ID NO: 10 - DNA, 2376 bp, Artificial
  HIV sequence inserted into delta 4-SHIV KU-2
cgcccgaacagggacgcgaaagcgaaagtagaaccagaggagctctctcgacgcaggactcggcttgctgaagcgcgcacggcaagaggcgaggggcggcgactggtgagtacgccatttttgactagcggaggctagaaggagagagatgggtgcgagagcgtcaatattaagcgggggacaattagatagatgggaaaaaattcggttacggccagggggaaagaaaagatataagttaaaacatatagtatgggcaagcagagagctagaacgattcgcagttaaccctggcctgttagaaacagcagaaggctgtagacaaatactgggacagctacaaccatcccttcagacaggatcagaggaacttaaatcattatttaatacaatagctaccctctattgtgtacatcaaagaatagagataaaagacaccaaggaagctttagataagatagaggaagagcaaaacaaaagtaagaaaaaagcacagcaagcagcagctgacacaggaaacagcagcagccaagtcagccaaaattaccctatagtgcagaacgctcagggacaaatggtacatcaggccatatcacctagaactttaaatgcatgggtaaaagtagtagaagaaaaggcttttaacccagaagtaatacccatgtttgcagcattgtcagaaggagccaccccacaagatttaaacaccatgctaaacacagtggggggacatcaagcagccatgcaaatattaaaagagactatcaatgaggaagctgcagaatgggatagattgcatccagtacatgcagggcctattgcaccaggccaaatgagagaaccaaggggaagtgacatagcaggaactactagtacccttcaggaacaaataggatggatgacaaataatccacctatcccagtaggagaaatctataaaaaatggataatcatgggattaaataaaattgtaaggatgtatagccctaccagtattctggacataagacaaggaccaaaggaaccctttagagactatgtagaccggttctataaaactctaagagccgagcaagcttcacaggaagtaaaaaattggatgacagaaaccttgttggtccaaaattcaaaccccgattgtaagactattttaaaagcattaggaccaggagctacactagaagaaatgatgacagcatgccagggagtgggaggacctggccataaagcaagagttttggcagaagcaatgagccaagtaacaaatccaacggccgtgatgatgcagaaaagcaattttaggggccaaagaaaaattgttaagtgttttaattgtggcaaagaagggcacatagccaaaaattgcagggctcctagaaaaaagggctgttggaaatgtggaaaggaaggacaccaaatgaaagattgtactgaaagacaggctaattttttagggaagatctggccttcctacaagggaaggccagggaattttcctcaaagcaggctagaaccaacagccccaccagaagcgagcttcaggtttggggaggagacaacaactccccctcagaagcaggagacgatagacaaggaggtgtatcctttaacctccctcagatcactctttggcaacgacccctcgtcacaataaagataggggggcaactaaaagaagctctattagatacaggagcagatgatacagtgttagaagacatgaatttgccaggaaaatggaaaccaaaaatgatagggggaattggaggatttatcaaagtaaaacagtatgatcagatacccatagaaatctgtggacataaaactataggtacagtattaataggacctacacctgtcaacataattggaaggaatttgttgactcagcttggttgcactttaaattttcccattagtcctattgaaactgtaccagtaaaattaaagccaggaatggatggcccaaaagttaagcaatggccattgacagaagaaaaaataaaagcattaatggagatatgcacagaaatggaaaaggaagggaaaatttcaaaaattgggcctgaaaatccatacaatactccagtgtttgccataaagaaaaaagacagtactaagtggagaaaattagtagatttcagagaacttaataagaaaactcaagacttctgggaggttcaattaggaataccacatcccgcggggttaaaaaagaaaaagtcagtaacagtactggatgtgggtgatgcatacttctcagttcccttagatgaagattttaggaagtatactgcatttaccatacctagtataaacaatgagacatcaggaattagatatcagtacaatgtgcttccacagggatggaaaggg

SEQ ID NO: 11 - DNA, 411 bp, Artificial; identical to SEQ ID NO: 4
  Nucleotide sequence deleted from 3′ LTR of SHIV KU-2

SEQ ID NO: 12 - DNA, 481 bp, Artificial
  SV40 polyadenlyation signal sequence inserted in delta 4-SHIV KU-2
tcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaa

SEQ ID NO: 13 - DNA, 818 bp, Artificial
  SIV promotor in delta 4-SHIV KU-2
tggaagggatttattacagtgcaagaagacatagaatcttagacatgtacttagaaaaggaaaaaggcatcataccagattggcaggattacacctcaggaccaggaattagatacccaaagacatttggctggctatggaaattagtccctgtaaatgtatcagatgaggcacaggaggatgaagagcattatttaatgcatccagctcaaacttcccagtgggatgacccttggagagaggttctagcatggaagtttgatccaactctggcctacacttatgaggcatatgttagatacccagaagagtttggaagcaagtcaggcctgtcagaggaagaggttaaaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcaggttgagccctgggaggttctctccagcactagcaggtagagcctgggtgttccctgctagactctcaccagcacttggccggtgctgggcagagtgattccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

(19) United States
(12) Patent Application Publication
(10) Pub. No.: US 2007/0010471 A1
(43) Pub. Date: Jan. 11, 2007

(54) HIV DNA vaccine

(76) Inventor: Opendra Narayan, Shawnee, KS (US)

Correspondence Address: POLSINELLI SHALTON WELTE SUELTHAUS P.C., 700 W. 47TH STREET, SUITE 1000, KANSAS CITY, MO, 64112-1802, US

(21) Appl. No.: 11/360,957

(22) Filed: Feb. 23, 2006

Related U.S. Application Data
(63) Continuation-in-part of application No. 10/279,992, filed on Oct. 24, 2002, now abandoned.
Application No. 10/279,992 is a continuation-in-part of application No. 08/850,492, filed on May 2, 1997, now abandoned.
(63) Continuation-in-part of application No. 10/941,164, filed on Sep. 15, 2004.
(60) Provisional application No. 60/503,197, filed on Sep. 16, 2003.

(51) Int. Cl. A61K 48/00; A61K 39/21

(52) U.S. Cl. 514/44; 424/208.1

(57) ABSTRACT

A DNA vaccines or immunogenic composition for providing an immune response against HIV without exhibiting pathogenicity in the immunized individual because of the disruption of the ability of the DNA molecules to encode for viral proteins critical in producing pathogenicity. The DNA molecule is derived by passaging a SHIV in order to develop a SHIV that exhibits an increased replication efficiency and increased pathogenicity. Following passaging, the highly virulent SHIV virus is rendered safe by disrupting one or more genes, such as the rt, int, and vif genes, as well as the 3′ LTR.

### CROSS-REFERENCE TO RELATED APPLICATIONS

This application is a Continuation-in-part of U.S. patent application Ser. No. 10/279,992 filed Oct. 24, 2002 entitled “HIV Vaccine and Method of Use” (which incorporates by reference U.S. patent application Ser. No. 08/850,492 filed on May 2, 1997, now abandoned) and is also a continuation-in-part application of Ser. No. 10/941,164 entitled “DNA Vaccine Compositions and Methods of Use” filed Sep. 15, 2004, which claims priority to a provisional application Ser. No. 60/503,197 filed on Sep. 16, 2003, all of which are incorporated herein by reference.

### STATEMENT REGARDING FEDERALLY SPONSORED RESEARCH OR DEVELOPMENT

This work was supported in part by NIH grant numbers 1 RO1 A151220-01 and 5 P20 RR16443-03. The government of the United States of America may have rights in this invention.

### BACKGROUND OF THE INVENTION

1. Field of the Invention

The present invention relates generally to the field of therapeutic and prophylactic immunogenic compositions and vaccines for generating protection from HIV-1 induced disease and infection. More specifically, the present invention relates to live virus and DNA vaccines against the Human Immunodeficiency Virus (“HIV”).

2. Description of Related Art

By the end of the year 2000, an estimated 36.1 million people worldwide were infected with HIV. In that year alone, HIV/AIDS-associated illnesses claimed the lives of approximately 3 million people worldwide. An estimated 500,000 of those deaths were of children under the age of fifteen. The importance of an HIV vaccine with respect to world health cannot be stated strongly enough.

It is recognized that effective vaccines that will inhibit or prevent HIV-1 infection or HIV-1-induced disease in humans will be useful for the treatment of certain high-risk populations, and as a general prophylactic vaccination for the general population that may risk HIV-1 infection or HIV-1-induced disease. A vaccine that will confer long-term protection against the transmission of HIV-1 would be most useful. Unfortunately, numerous problems stand in the way of developing effective vaccines for the prevention of HIV-1 infection and disease. Certain problems are most likely the result of the unique nature of the HIV-1 virus and its functional properties, and as yet no effective vaccine has been developed (for review see: Berzofsky et al., Developing Synthetic Peptide Vaccines for HIV-1, Vaccines 95, pps. 135-142, 1995; Cease and Berzofsky, Toward a Vaccine for AIDS: The Emergence of Immunobiology-Based Vaccine Design, Annual Review of Immunology 12:923-989, 1994; Berzofsky, Progress Towards Artificial Vaccines for HIV, Vaccines 92, pps. 41-40, 1992).

HIV is a retrovirus, meaning that its genome consists of RNA rather than DNA. There are two primary strains of the virus, designated HIV-1 and HIV-2, with HIV-1 being the strain that is primarily responsible for human infection. The RNA genome of HIV is surrounded by a protein shell. The combination of the RNA genome and the protein shell is known as the nucleocapsid, which is in turn surrounded by an envelope of both protein and lipid.

Infection of host cells by HIV begins when the gp120 protein of HIV, a highly glycosylated protein located in the viral envelope, binds to the CD4 receptor molecule of the host cell. This interaction initiates a series of events that allow fusion between the viral and cell membranes and the subsequent entry of the virus into the cell.

Following entry into the host cell, HIV RNA is transcribed into double-stranded DNA by a viral reverse transcriptase enzyme. The HIV DNA is then integrated into the host cell genome by the action of the viral integrase enzyme. Once integrated into the host genome, HIV expresses itself through transcription by the host's RNA Polymerase II enzyme. Through both transcriptional control and postranscriptional transcript processing, HIV is able to exert a high level of control over the extent to which it expresses itself.

Studies of the HIV virus have revealed much information about the molecular biology of the virus, including information concerning a number of genes important to the pathogenicity of HIV. Such genes include rt, int, vif, gag, pol, nef, and vpu genes, and the 3′ Long Terminal Repeat (“LTR”) of HIV.

The rt gene of HIV encodes the viral reverse transcriptase enzyme. This enzyme utilizes the RNA genome of HIV to produce a corresponding linear double-stranded DNA molecule that can be incorporated into the host genome.

The int gene of HIV encodes the integrase protein. This is the enzyme that catalyzes the insertion of the reverse-transcriptase-produced linear double-stranded viral DNA into the host genome. In order to complete integration of the viral DNA into the host genome, the host cell DNA repair machinery performs a ligation of the host and viral DNAs.

The vif gene of HIV encodes a protein known as the “viral infectivity factor.” This protein is required for the production of infectious virions. The protein likely overcomes a cellular inhibitor that otherwise inhibits HIV-1, and may also enhance the stability of the viral core and the preintegration complex.

The gag gene encodes for, among other things, the p27 capsid protein of HIV. This protein is important in the assembly of viral nucleocapsids. The p27 protein is also known to interact with the HIV cellular protein CyA, which is necessary for viral infectivity. Disruption of the interaction between p27 and CyA has been shown to inhibit viral replication.

The pol gene encodes viral enzymes important in enabling the virus to integrate into the host genome and replicate itself. The pol gene encodes, among other proteins, viral reverse transcriptase (“RT”) and integrase (“IN”).

The nef gene product (known as Negative Factor or Nef) has a number of potentially important properties. Nef has the ability to downregulate CD4 and MHC Class I proteins, both of which are important to the body's ability to recognize virus-infected cells. Nef has also been shown to activate cellular protein kinases, thereby interfering with the signaling processes of the cell. Perhaps most importantly, deletion of the nef gene from a pathogenic clone of Simian Immunodeficiency Virus (“SIV”) renders the virus nonpathogenic in adult macaque monkeys. Thus, a functional nef gene is crucial for the ability of SIV to cause disease in vivo. Further, studies have shown that HIV positive individuals with large deletions in the nef gene remained healthy for well over 10 years, with no reduction in cellular CD4 counts.

The vpu gene encodes a protein of originally unknown function (known as Viral Protein, Unknown, or Vpu), but which is now known to downregulate CD4 and MHC Class-I expression as well as promote viral budding. Vpu is also similar to another viral protein that acts as an ion channel. The vpu gene is present in HIV-1, but is absent in HIV-2.

The LTR regions of HIV-1 contain promoter regions necessary to drive expression of the HIV genes. The 5′ LTR of HIV-1 contains the promoter that is primarily responsible for driving HIV-1 gene expression, though if the 5′ LTR sequence is disrupted, the 3′ LTR may assume this function. The 3′ LTR is necessary for integration of the viral DNA into the host genome.

In nearly all viral infections, certain segments of the infected population recover and become immune to future viral infection by the same pathogen. Examples of typical viral pathogens include measles, poliomyelitis, chicken pox, hepatitis B, small pox, etc. The high mortality rate of HIV-1 infection, and the extremely rare incidence of recovery and protective immunity against HIV-1 infection, has cast doubt on the ability of primates to generate natural immunity to HIV-1 infection when pathogenic HIV-1 is the unmodified wild-type viral pathogen. Thus, there is a great need for a vaccine that will confer on primate populations, protective immunity against HIV-1 virus.

One possibility for such a vaccine could come in the form of a DNA vaccine against HIV-1. DNA vaccines are generally injected into host tissues in the form of plasmid DNA or RNA molecules via needle or particle bombardment. Once delivered, the DNA induces expression of antigenic proteins within transfected cells. U.S. Pat. No. 6,194,389 describes methods for transferring DNA to vertebrate cells to produce physiological immune-response producing protein in an animal subject and is incorporated herein in its entirety by reference.

Testing of vaccine efficacy requires inoculation then challenge of the subject with DNA vaccine. Of course, it is ethically and practically difficult to attempt preliminary studies using human subjects. The use of model systems for preliminary design and testing of candidate vaccines has been hampered by various species-specific features of the virus. The HIV-1 virus itself is currently known only to infect certain rare and endangered species of chimpanzee in addition to humans. The feasibility of obtaining sufficient numbers of such endangered animals for full preliminary study of HIV-1 virus vaccines is quite low. It is preferable to use validated analogous animal model systems.

One analogous model system for HIV-1 infection has been the Simian Immunodeficiency Virus, macaque (“SIV_{mac}”) system. SIV infects a variety of simians, including macaques, but the differences between SIV and HIV make SIV of limited used as a potential human vaccine.

In addition, a chimeric SIV-HIV virus has been developed by placing the envelope proteins of HIV-1 on a background of SIV_{mac}. The chimeric virus proved to be infectious to monkeys, but did not result in full-blown AIDS or an accurate model to mimic HIV-1 infection in monkeys (see generally Shibata and Adachi, SIV/HIV Recombinants and their use in Studying Biological Properties, AIDS Research and Human Retroviruses 8(3):403-409, 1992; Sakuragi et al., Infection of Macaque Monkeys with a Chimeric Human and Simian Immunodeficiency Virus, J. General Virology, 73:2983-2987, 1992).

An improved SHIV chimeric virus known as SHIV-4 was derived from the HIV HXBc2 strain as described in Li et al., Infection of cynomolgus monkeys with a chimeric HIV-1/SIVmac virus that expresses the HIV-1 envelope glycoproteins. J. Acquired Immune Defic. Syndr. 5:639-646 (1992) and Sodroski et al., “Hybrid SIV/HIV-1 Viral Vectors and Monkey Model for AIDS,” WO 93/24632. This SHIV-4 virus was later passaged in rhesus and pig-tailed macaques to develop the non-pathogenic SHIV_{ppc} virus. Additional passaging in pig-tailed macaques resulted in the highly replication-efficient, more virulent SHIV viral population, known as SHIV_{KU-1}, which is the subject of Narayan, U.S. Pat. No. 5,849,994 entitled “Animal Model for HIV-1 Induced Disease.” Further passaging in rhesus monkeys resulted in SHIV_{KU-2}, as described in Ser. No. 08/850,492, now abandoned (see also WO 98/50070), and incorporated by reference by parent patent application Ser. No. 10/279,992.

With these animal models in hand, efforts in HIV vaccine research turned to the creation of various chimeric SHIV vaccines. Many of these proposed vaccines were live virus vaccines. For example, Narayan, WO 98/50070 entitled “Live Virus Vaccines to Protect Primates from HIV-1 Infection and Disease,” describes a live virus vaccine in which the vpu and nef genes associated with the cDNA clone of the non-pathogenic SHIV_{ppc} are deleted to yield ΔvpuSHIV_{ppc} and ΔvpuΔnefSHIV_{ppc}. In both instances, macaques were inoculated with the live infectious viral particle as a vaccine. After inoculation with of the live viral vaccine, the animals are challenged with the pathogenic SHIV_{KU-1} virus.

The present invention does not rely on a live virus vaccination strategy. Instead, the present invention is a directed to an improved “DNA vaccine” or “nucleic acid vaccine” which involves the direct injection of a genes coding for a specific antigenic proteins, resulting in direct production of such antigens within the vaccine recipient in order to trigger an appropriate immune response. The DNA construct used as the basis for the vaccine is based on a high-efficiency, pathogenic SHIV virus rendered safe by deletion of the rt gene.

### BRIEF SUMMARY OF THE INVENTION

The present invention is directed to DNA vaccines for providing an immune response against HIV without exhibiting pathogenicity in the immunized individual because of the disruption of the ability of the DNA molecules to encode for viral proteins critical in producing pathogenicity. More specifically, the present invention is directed to a DNA SHIV vaccine in which the rt gene is deleted to eliminate the ability of the virus encoded by the DNA to make a DNA copy of the RNA genome. As such, the DNA molecule of the present invention produces viral particles within the host cell, but such viral particles are non-pathogenic. The antigen presenting cells of the immune system process these viral particles, which lead to the development of an antiviral immune response. In addition, the infected cell can produce these non-pathogenic viral particles to provide long-term viral protection.

In the present invention, it is surprisingly discovered that an improved non-pathogenic, non-infectious DNA vaccine can be constructed by increasing the pathogenicity of the SHIV virus by passaging prior to rendering the SHIV virus safe by deletion of the rt gene to render the virus safe and non-pathogenic. In an exemplary embodiment, the parent SHIV-4 virus is subjected to serial passages to create the highly pathogenic and efficiently replicating SHIV_{KU-2} virus, which in turn is rendered safe by the deletion of one or more genes, such as the rt, int, and vif genes, as well as the 3′ LTR. By using the using the high-efficiency SHIV_{KU-2} as the basis for the non-infectious, non-pathogenic DNA vaccine, the vaccine demonstrates an improved ability to cause an immune response. It is theorized that the DNA vaccine enters the cell and replicates efficiently to produce more viral antigens because of the nature of the SHIV_{KU-2} genome and highly efficient promoter. However, because the rt, int, and vif have been deleted, a functional infectious viral particle cannot be assembled, rendering the vaccine safe.

In one aspect, the present invention is directed to a DNA molecule derived by at least two successive passages in vivo of a SHIV viral isolate through macaque bone marrow that is subsequently rendered non-pathogenic by deletion of one or genes critical to the viral infectivity and pathogenicity. The passaging preferably results in a SHIV that is infectious in monkeys and causes a monkey to develop AIDS-associated symptoms within about 32 weeks of infection.

In another aspect, the DNA molecule provides protective immunity against HIV by encoding the plurality of viral proteins capable of stimulating an immune response is selected from a group of coding sequences comprising the gag, pro, tat, rev, vpu, env, vpx, vpr and nef genes of either SIV or HIV.

In still another aspect, the DNA molecule is selected from the group consisting of SEQ ID NO: 1 (ΔrtSHIV_{KU-2} or V5), SEQ ID NO: 3 (ΔrtΔ3′LTRSHIV_{KU-2} or V6), SEQ ID NO: 5 (ΔrtΔ3′LTRSHIV_{KU-2} or V7), and SEQ ID NO: 7 (Δ4-SHIV_{KU-2}).

Additional aspects of the invention, together with the advantages and novel features appurtenant thereto, will be set forth in part in the description that follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 is a diagram showing the generation of pathogenic, replication-efficient SHIV, namely SHIV_{KU-1} and SHIV_{KU-2}.

FIG. 2 is a diagram showing the construction of plasmid-based vaccine pET-9a/ΔrtSHIV_{KU-2}, also referred to herein as the V5 embodiment of the present invention.

FIG. 3 is a diagram showing the construction of plasmid-based vaccine pET-9a/ΔrtΔ3′LTR SHIV_{KU-2}, also referred to as the V6 embodiment of the present invention.

FIG. 4 is a diagram showing the construction of the plasmid-based vaccine referred to as the V7 embodiment of the present invention.

FIG. 5 is a schematic diagram showing the schematic layouts of the V5, V6, and V7 embodiments of the present invention, as well as the schematic layout of a vector of the present invention.

FIG. 6 is a schematic diagram of the Δ4-SHIV_{KU-2} DNA construct of the present invention.

FIG. 7 is a circular diagram of the Δ4-SHIV_{KU-2} DNA construct of the present invention.

### DETAILED DESCRIPTION OF PREFERRED EMBODIMENT

The present invention is directed to DNA vaccines for providing an immune response against HIV without exhibiting pathogenicity in the immunized individual because of the disruption of the ability of the DNA molecules to encode for viral proteins critical in producing pathogenicity. The DNA molecule is derived by passaging a SHIV in order to develop a SHIV that exhibits an increased replication efficiency and increased pathogenicity. Following passaging, the highly virulent SHIV virus is rendered safe by disrupting one or more genes, such as the rt, int, and vif genes, as well as the 3′ LTR.

A better understanding of the present invention may be obtained in light of the following examples that are set forth to illustrate, but are not to be construed to limit the present invention.

### EXAMPLE 1

### Generation of Pathogenic SHIV

In this example, the generation of a SHIV having increased pathogenicity by serial passaging is described. The exemplary passaged SHIV generates full-blown AIDS in monkeys in a relatively short period of time. The exemplary pathogenic viruses, named SHIV_{KU-1} and SHIV_{KU-2} (originally isolated from animals PPc/14a and PNb), are the first virus bearing the envelope of HIV-1 that can cause AIDS in a non-human primates.

The pathogenicity of the SHIV is demonstrated by the fact that (a) all animals lost CD4⁺ T Cells during the first three weeks after inoculation with the virus (an excellent marker for virus pathogenicity); (b) the virus is predictably pathogenic, with 70% of inoculated animals developing AIDS within six months (and thus vaccine efficacy can be evaluated in a short time using this monkey model system); and (c) the virus invades across mucosal surfaces and causes AIDS after deposition in the mouth or in the vagina (thus allowing for evaluation of testing for efficacy against sexual transmission).

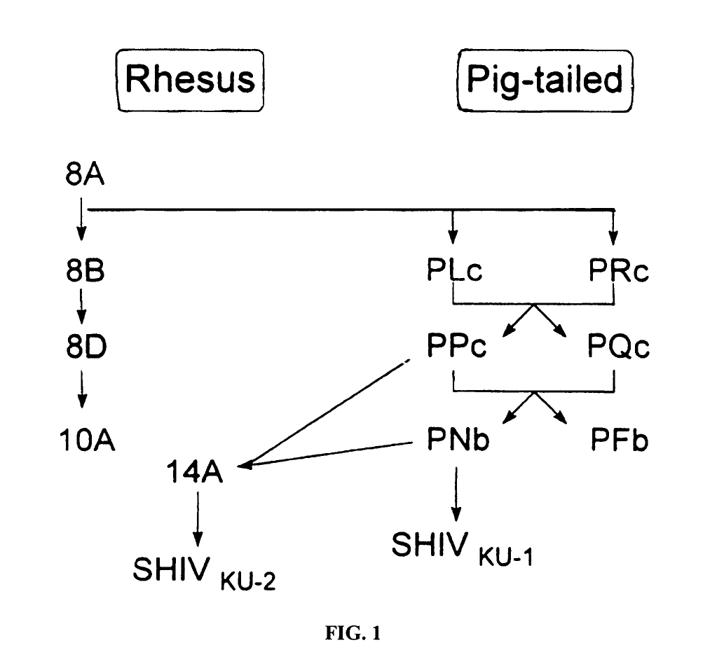

Development of pathogenic SHIV_{KU-1} is described in Narayan, U.S. Pat. No. 5,849,994. SHIV_{KU-1} was derived by sequential passage of a virus through bone marrow using an SHIV construct containing tat, rev, vpu, and env genes derived from a laboratory strain of HIV-1 obtained from Dr. Joesph Sodroski, Harvard University (see also Joag et al., Chimeric Simian/Human Immunodeficiency Virus That Causes Progressive Loss of CD4⁺ T Cells and AIDS in Pig-Tailed Macaques, J. Virology 70(5):3189-3197, 1996). For the first passage (Passage 1), 1×10⁴ TCID (tissue culture infective dose) of SHIV virus was inoculated into the bone marrow (“BM”) of rhesus macaque 8A. Five weeks later, heparinized BM was obtained from this animal, mononuclear cells were purified over Ficoll-Hypaque gradients and 5×10⁷ cells were inoculated into the femoral bone marrow of two pig-tailed macaques, PLc and PRc (Passage 2). Five weeks later, BM was aspirated from PLc and PRc (2 ml each), pooled, purified as above, and inoculated into the BM of two new pig-tailed macaques, PPc and PQc (Passage 3). Sixteen weeks later, bone marrow and splenic biopsies were obtained from macaques PPc and PQc, and a mixture of splenocytes and BM cells from both animals were pooled and inoculated into two new pig-tailed macaques, PFb and PNb (Passage 4). As shown in FIG. 1, the virus recovered from the PNb pig tailed macaque is known as SHIV_{KU-1}.

The derivation of the virulent SHIV_{KU-2} variant is described in U.S. patent application Ser. No. 08/442,010, now abandoned, entitled “Live Virus Vaccine to Protect Primates from HIV-1 Infection and Disease” filed on May 2, 1997, which is incorporated by reference in parent patent application Ser. No. 10/279,992 entitled “HIV Vaccine and Method of Use” filed on Sep. 24, 2002, and is also incorporated by reference herein. More specifically, as shown in FIG. 1, virus from PPc and Pnb was pooled and passaged an additional time through a Rhesus macaque (animal 14A) now named SHIV_{KU-2}. The viral passaging history of SHIV_{KU-1} and SHIV_{KU-2} is summarized in FIG. 1. The virus isolated from animal PPc, a pig-tailed macaque was not pathogenic.

The pathogenicity of the SHIV_{KU-1} virus is described generally in U.S. Pat. No. 5,849,994 entitled “Animal Model for HIV-1 Induced Disease”; Stephens et al., “A cell-free stock of simian-human immunodeficiency virus that causes AIDS in pig-tailed macaques has a limited number of amino acid substitutions in both SIV_{mac} and HIV-1 regions of the genome and has offered cytotropism,” Virology, 231:313-321 (1997); Joag et al., “Animal model of mucosally transmitted human immunodeficiency virus type 1 disease: intravaginal and oral deposition of simian/human immunodeficiency virus in macaques results in systemic infection, elimination of CD4+ T cells, and AIDS,” J. Virol., 71:4016-4023 (1997); Joag et al., “Passively administered neutralizing serum that protected macaques against infection with parenterally inoculated pathogenic simian-human immunodeficiency virus failed to protect against mucosally inoculated virus,” AIDS Res. Hum. Retroviruses, 15:391-394 (1999); Joag et al., “Oral immunization of macaques with attenuated vaccine virus induces protection against vaginally transmitted AIDS,” J. Virol., 72:9069-9078 (1998); and Silverstein et al., “Pathogenic simian/human immunodeficiency virus SHIV(KU) inoculated into immunized macaques caused infection, but virus burdens progressively declined with time,” J. Virol., 74:10489-10497 (2000), all of which are incorporated by reference. The replication of SHIV_{KU-1} in human PBMC cultures is shown the table below:

TABLE 1
Replication of SHIV_{KU} in Human PBMC Culture (pg/ml of p27)

| | | Control | SHIV_{KU} |
|---|---|---|---|
| Day | 0 | Neg | Neg |
| | 2 | Neg | 525 |
| | 4 | Neg | 12,466 |
| | 6 | Neg | 18,117 |
| | 8 | | 17,988 |
| | 10 | | 15,909 |

The experiment utilized 2×10⁶ PHA stimulated PBMCs. The inoculum was 2000 TCID₅₀ of each virus.

The pathogenicity SHIV_{KU-2} is described generally in Liu et al., “Derivation and biological characterization of a molecular clone of SHIV(KU-2) that causes AIDS, neurological disease, and renal disease in rhesus macaques,” Virology, 260:295-307 (1999) and Foresman et al., “Neutralizing antibodies administered before, but not after, virulent SHIV prevent infection in macaques,” AIDS Res. Hum. Retroviruses, 14:1035-1043 (1998); and Raghavan et al., “Neuropathogenesis of chimeric simian/human immunodeficiency virus infection in pig-tailed and rhesus macaques,” Brain Pathol. July; 7(3):851-61 (1997), all of which are incorporated by reference.

The pathogenicity of SHIV_{KU-1} and SHIV_{KU-2} is also generally described in parent patent application Ser. No. 10/279,992 entitled “HIV Vaccine and Method of Use”, which is incorporates by reference patent application Ser. No. 08/850,492, now abandoned.

### EXAMPLE 2

### HIV Vaccine: ΔrtSHIV_{KU-2} or “V5”

### EXAMPLE 2B

### Construction of ΔrtSHIV_{KU-2} or “V5”

In this example, the passaged SHIV from Example 1 having increased pathogenicity is used to create a safe and effective vaccine by deleting the rt gene. This example is described in Example 8 of parent patent application Ser. No. 10/279,992 entitled “HIV Vaccine and Method of Use” filed on Sep. 24, 2002.

More specifically, the rt gene was deleted in a passaged, highly pathogenic SHIV virus to create a novel vaccine. The SHIV was utilized to develop a DNA vaccine that provides transfected cells with the ability to shed viral proteins into the extracellular environment while retaining a safety and efficacy. As discussed above, SHIV_{KU-1} was shown to be highly efficient in replication in macaques and human PBMC cultures. In addition, SHIV_{KU-1} has a high degree of pathogenicity in macaques. Rapid replication of the virus causes subtotal elimination of the CD4+ T cell population within a few weeks of infection (as described above). In addition, when administered to animals that have been previously immunized with vaccine viruses, SHIV_{KU-1} induces a potent ananmestic immune response that is associated with the development of curative immunity against the virus (see Silverstein et al., “Pathogenic simian/human immunodeficiency virus SHIV(KU) inoculated into immunized macaques caused infection, but virus burdens progressively declined with time,” J. Virol., 74:10489-10497 (2000)). The high replicative efficiency of the virus was found to be associated with enhanced transcription of viral RNA, which in turn appears to be mediated by a unique interaction between Nef, the transcription factor NFAT, and sequences in the U3 region of the viral promoter. Further, the DNA of SHIV_{KU-1} exhibited better persistence in the lymph nodes of challenged animals than did the DNA of SHIV 89.6P and SIV. The ability of this DNA to persist in the lymph nodes, in addition to its enhanced capacity for expressing viral proteins, is a major asset in the efficacy of the DNA as a DNA vaccine.

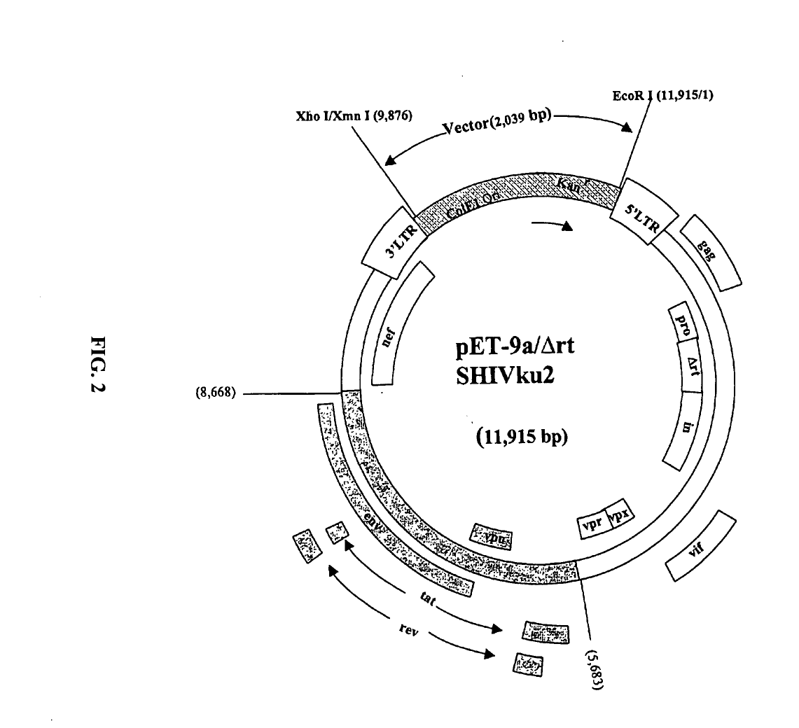

The SHIV was passaged an additional time as set forth in FIG. 1 to obtain the pathogenic SHIV_{KU-2}. In order to render this embodiment of the present invention safe, the sequences encoding reverse transcriptase were removed, resulting in the ΔrtSHIV_{KU-2} DNA vaccine (the V5 vaccine). A schematic diagram of the V5 vaccine DNA construct is provided in FIG. 2. As can be seen in FIG. 2, the vector used for this embodiment of the present vaccine is pET-9a. The 2.3 kb EcoRI/XmnI fragment of the plasmid was replaced by the 9.88 kilobase provirus genome of SHIV_{KU-2}. An EcoRI restriction site was created immediately upstream of the 5′ LTR and an XhoI restriction site was created immediately at the end of the 3′ LTR. The sequence of the V5 DNA vaccine is provided in SEQ ID NO:1. The rt sequence was disrupted by deletion of 762 base pairs, while the protease and integrase genes were left intact. The precise deletions made in the rt are set forth in SEQ ID NO:2. The precise location of the sequence within the rt gene can be readily determined as the rt gene sequence is known and the location of the deleted sequence can be determined manually to via any computer program designed to align DNA sequences. It is understood, of course, that any modification to the rt gene sufficient to disrupt its functionality is acceptable. The disruption of the gene may even include a full deletion of the rt gene. The size of the construct is 11,915 base pairs, being composed of a 2033 base pair vector and the 9882 base pair provirus genome.

The following examples detail experimental studies performed with the ΔrtSHIV_{KU-2} vaccine.

### EXAMPLE 2B

### Transfection of ΔrtSHIV_{KU-2} (V5) into CEM 174 Cells

This example is described in Example 9 of parent patent application Ser. No. 10/279,992 entitled “HIV Vaccine and Method of Use” filed on Sep. 24, 2002. Five μg of ΔrtSHIV_{KU-2} (V5) DNA was transfected into approximately 2×10⁶ CEM 174 cells. The transfected cell cultures developed fusion CPE on day four following transfection. Supernatant fluid was collected from the culture at two-day intervals and the viral p27 content of the supernatant fluid was assessed. After each collection of supernatant fluid, the cell cultures were washed and placed in fresh medium to ensure that each two-day sample contained only viral p27 produced during the preceding two-day period. Approximately 3050 pg of viral p27 was detected in the supernatant fluid on day four. The V5 cultures became negative by day ten. Decline in viral protein production coincided with the disappearance of the syncytial cells from each culture, presumably by apoptotic mechanisms because the cell culture system utilized is highly susceptible to viral-induced fusion CPE. Importantly, most of the viral p27 observed was located in the supernatant fluid. The ability of the V5 transfected cells to shed viral proteins into the extracellular environment provides an opportunity for other cells to present viral antigens. Therefore, in addition to the ability of ΔrtSHIV_{KU-2} to cause enhanced transcription of its RNA and produce more viral proteins, the ability to shed viral proteins into the extracellular environment provides an added advantage.

### EXAMPLE 2C cl The Safety and Efficacy of ΔrtSHIV_{KU-2} (V5)

This example is described in Example 10 of parent patent application Ser. No. 10/279,992 entitled “HIV Vaccine and Method of Use” filed on Sep. 24, 2002. Portions of the ΔrtSHIV_{KU-2} supernatant fluid containing viral p27 and described above were inoculated into fresh cultures of CEM 174 cells. These new CEM 174 cells did not develop CPE and the supernatant fluids from these cultures lacked the molecules necessary to code for infectious viral particles. Thus, it was determined that the ΔrtSHIV_{KU-2} embodiment of the present invention is safe and is unable to produce infectious, pathogenic viral particles.

In order to determine whether the proteins expressed by the V5 vaccine virus would indeed be recognized by antibodies from an HIV-infected person, as well as by antibodies from previously immunized macaques, cell cultures infected with the vaccine virus and HIV-1, respectively, were pulsed with ³⁵S-labelled methionine, and then lysed and immunoprecipitated with serum from a long-term non-progressor with HIV infection as well as with serum from a macaque that had been previously immunized with the SHIV vaccine virus. Both of these sera bound the Env and Gag of both HIV and SIV in the infected cultures.

### EXAMPLE 3

### Construction of HIV Vaccine: Δrt Δ3′LTRSHIV_{KU-2} with SV40 Poly A Tail and SIV Nef or “V6”

In this example, the passaged SHIV virus having increased pathogenicity is used to create a safe and effective vaccine by deleting the rt gene, as well as the 3′ LTR. This example is described in Example 10 of Ser. No. 10/279,992 entitled “HIV Vaccine and Method of Use” filed on Sep. 24, 2002. More specifically, the rt gene and 3′ LTR was deleted in the passaged, highly pathogenic SHIV_{KU-2} virus were made to create a novel vaccine.

The 3′ LTR HIV are necessary for proper integration of the virus into the host genome. Eliminating the 3′LTR provides a virus that is unable to integrate into the host genome, while retaining the ability to encode for immunogenic viral proteins without encoding for infectious, pathogenic virus. This decreases the likelihood that the vaccine DNA will become inserted into a host oncogene, thereby causing oncogenesis. Thus, an additional embodiments of the present vaccine, known as the ΔrtΔ3′LTR SHIV_{KU-2} (V6) embodiment, was created.

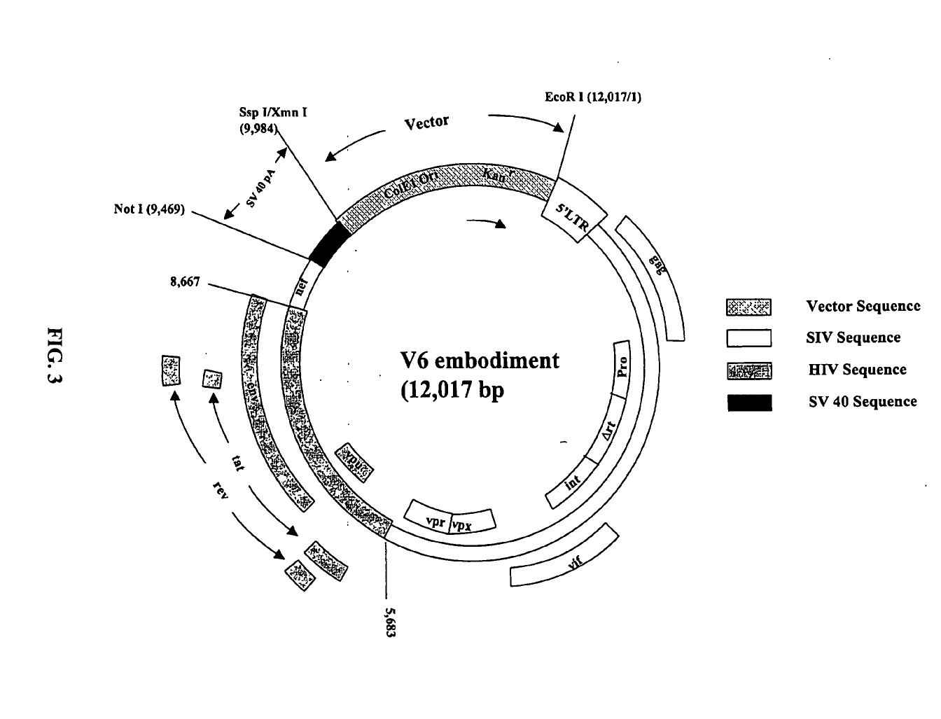

A schematic diagram of the pET-9a/ΔrtΔ3′LTR SHIV_{KU-2} (V6) vaccine DNA construct is provided in FIG. 3. The V6 vaccine represents an alternative embodiment of the present invention. The sequence of the V6 embodiment of the present invention is provided in SEQ ID NO:3. As can be seen in FIG. 3, the vector used for this embodiment of the present vaccine is pET-9a. The 2.3 kb EcoRI/XmnI fragment of the plasmid was replaced by the SHIV_{KU-2} provirus genome and a 515 bp SV40 polyadenylation sequences. An EcoRI restriction site was created immediately upstream of the 5′ LTR, and SV 40 polyadenylation sequences were added to the end of the nef gene. The rt gene was eliminated by the deletion of a 762 bp sequence, while the genes coding for viral protease and integrase were left intact. The precise 762 bp sequence deleted from the rt gene is the same as that deleted in the V5 embodiment of the present invention as provided in SEQ ID NO:2. The 3′ LTR was also disrupted, but only through a partial deletion due to the overlap of the 3′ LTR with the nef gene. The precise sequence of bases deleted from the 3′ LTR is provided in SEQ ID NO:4. Although FIG. 3 shows the V6 embodiment of the present invention as having an SIV nef gene, it is contemplated that the vaccine could alternatively have a nef gene derived from HIV, as discussed below in Example 3.

### EXAMPLE 4

### Construction of HIV Vaccine: Δrt Δ3′LTRSHIV_{KU-2} with SV40 Poly A Tail and HIV Nef or “V7”

### EXAMPLE 4A

### Construction of V7

This example is described in Example 10 of parent patent application Ser. No. 10/279,992 entitled “HIV Vaccine and Method of Use” filed on Sep. 24, 2002. More specifically, the rt gene and 3′ LTR was deleted in the passaged, highly pathogenic SHIV_{KU-2} virus were made to create a novel vaccine. In addition, the SV40 polyadenylation sequence is inserted into the genome. The vaccine is designated as the V7 embodiment or Δ2-SHIV_{KU-2}.

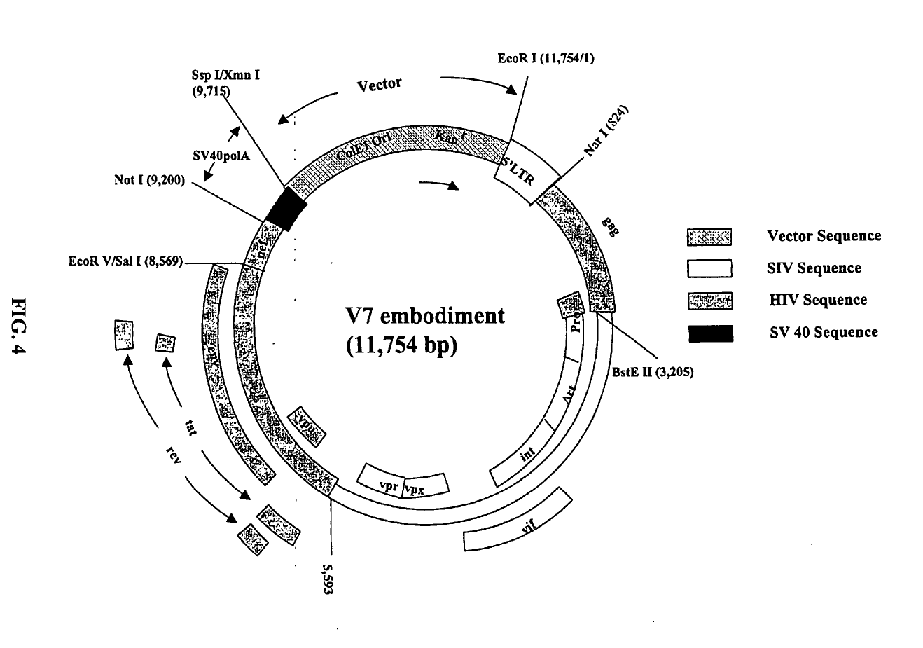

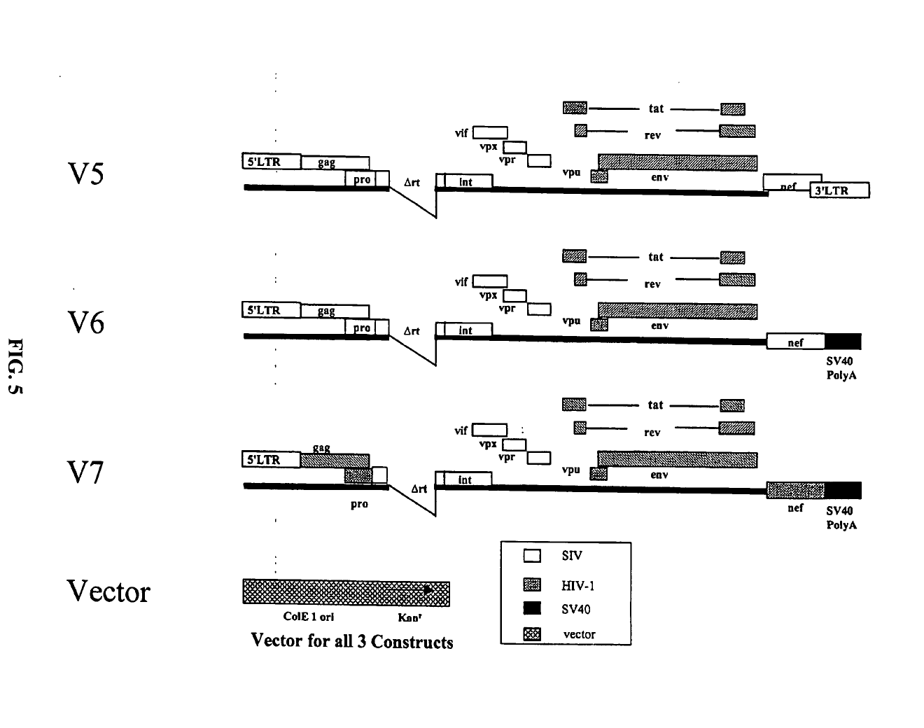

The sequence of the V7 embodiment of the present invention is provided in SEQ ID NO:5. A schematic diagram of the V7 embodiment of the present invention is provided in FIG. 4. The vector used is pET-9a. The 2.3 kb EcorI/Xmn I fragment of the plasmid was replaced by the SHIV_{KU-2} provirus genome and SV 40 polyadenylation sequences. The rt gene was disrupted by deletion of an 818 bp sequence, while the protease and integrase genes were kept intact. The precise 818 bp sequence deleted from the rt gene is the same as that deleted in the V5 embodiment of the present invention provided in SEQ ID NO:2 above. The sequence of the deleted 3′ LTR of the V7 embodiment is provided in SEQ ID NO:6.

### EXAMPLE 4B

### Efficacy of V7

Example 2 of parent patent application Ser. No. 10/941,164 entitled “DNA Vaccines and Methods of Use” filed on Sep. 15, 2004 describes the efficacy of the V7 Vaccine. More specifically, it is known from previous studies conducted by the inventor of the present invention that a live virus vaccine against HIV is highly efficient in eliciting protection against the virus. To establish that a DNA vaccine could be just as efficient in providing such protection, an experiment utilizing five macaques was conducted. Three of the animals were injected with the V7 DNA. The remaining two animals were immunized with the live virus vaccine ΔvpuΔnefSHIV_{ppc}. The three animals vaccinated with the DNA vaccine were each given 2 mg of the DNA, injected intradermally, followed by an intramuscular injection of 5 mg of DNA six weeks later, and a third, 0.5 mg intramuscular DNA injection twelve weeks later. The macaques were challenged intravenously with an undiluted stock preparation of SHIV 89.6P twelve weeks after the final immunization. It is important to note that the same dose of the SHIV 89.6P causes disease in 100% of inoculated control animals. The two macaques vaccinated with live virus were challenged ten weeks post-vaccination with the same SHIV virus.

When the animals were subsequently studied, it became clear that the DNA vaccine induces ELISPOT™ (Cellular Technology Limited, Cleveland, Ohio) responses against epitopes in the Env and Gag peptides, as well as neutralizing antibodies to SHIV_{KU-2}. ELISPOT™ responses are hereby defined as measures of the number of cells expressing an indicated epitope. All three animals vaccinated with the DNA vaccine became infected with SHIV 89.6P, but each developed only low levels of viral RNA in plasma, with no loss of CD4 T-cells. The animals vaccinated with the DNA vaccine V7 developed a massive anamnestic ELISPOT™ response following challenge.

The infection in these animals has been controlled for more than 28 weeks. At the 28-week point, the three animals that were immunized with DNA vaccine demonstrated protection that was as efficient as animals immunized with the live vaccine. Thus, the DNA vaccine proved to be just as efficient as the live vaccine in eliciting protection against heterologous SHIV 89.6P. Further, the animals receiving the DNA vaccination did not have to bear the burden of prior infection with a live vaccine virus.

### EXAMPLE 5

### Construction of HIV Vaccine: Δ4-SHIV_{KU-2}

### EXAMPLE 5A

### Construction of the Δ4-SHIV_{KU-2} DNA Construct

This example is described in Example 1 of Ser. No. 10/941,164 entitled “HIV Vaccine and Method of Use.” More specifically, the rt gene, int gene, vif gene, and 3′ LTR was deleted in the passaged, highly pathogenic SHIV_{KU-2} virus were made to create a novel vaccine. The vaccine is designated as Δ4-SHIV_{KU-2}.

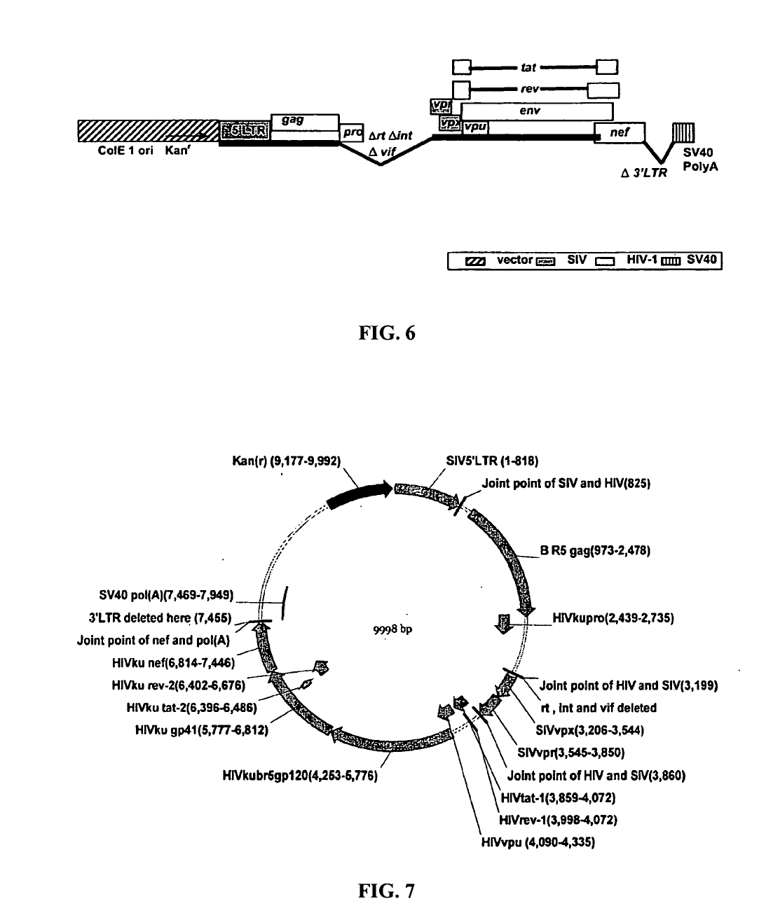

FIG. 6 is a schematic diagram of the Δ4-SHIV_{KU-2} DNA construct. The construction of the Δ4-SHIV_{KU-2} DNA construct (SEQ ID NO:7) is performed as follows. The vector used for the present vaccine is pET-9a. The 2.3 kb EcoR I/Xmn I fragment of pET-9a is replaced with the approximately 7.4 kb modified SHIV_{KU-2} provirus genome and the approximately 0.5 kb polyadenylation signal sequence of SV40 to yield an intermediate vector. EcoRI and Not I restriction sites are created immediately upstream of the 5′ LTR and at the end of the nef gene, respectively, in another intermediate vector. The reverse transcriptase (rt), integrase (int), and vif genes are eliminated by deletion of an approximately 2.5 kb DNA fragment between the downstream end of the pro gene and upstream of the vpx gene. The approximately 3.8 kb nucleotide sequence that encodes the envelope (env), nef, and 3′ LTR genes of SHIV_{KU-2} provirus genome is then replaced with the approximately 3.2 kb EcoRV/Not I DNA fragment that encodes the env and nef genes of HIV-1. The approximately 2.5 kb Nar I/BstE II DNA fragment that encodes the leader sequence, gag, and pro genes of SIV_{mac239} in SHIV_{KU-2} is replaced with an approximately 2.4 kb Nar I/BstE II fragment that encodes the HIV-1 leader sequence, gag, and pro of HIV-1 to yield Δ4-SHIV_{KU-2} DNA construct (SEQ ID NO:7). Thus, the 5′ LTR, vpx, and vpr genes of the present vaccine are from SIV_{mac239}, and the gag, pro, tat, rev, vpu, env, and nef are from HIV-1. The sequence of a preferred embodiment of the present DNA vaccine Δ4-SHIV_{KU-2} DNA is designated SEQ ID NO:7.

The information below is provided to detail structure of the Δ4-SHIV_{KU-2} DNA construct (SEQ ID NO:7) more completely. A 4,981 bp fragment of SHIV_{KU-2} that encodes the entire gag, and pol genes (which therefore includes the rt and int portions of the genome), as well as the first 472 bp of the vif gene, is replaced with a 2,376 bp DNA fragment of HIV-1 in the Δ4-SHIV_{KU-2} DNA construct. This 2,376 bp fragment encodes the entire HIV-1 gag gene, and a portion of the HIV-1 pol gene (the entire region encoding protease is included; the nucleotides corresponding to the first 104 amino acids of reverse transcriptase have been removed; the int and vif genes have been completely removed. The 4,981 bp fragment of SHIV_{KU-2} that was replaced is designated SEQ ID NO:8. The DNA sequence of the first 472 bp of the vif gene of SHIV_{KU-2}, which was also replaced is designated SEQ ID NO:9. The DNA sequence of the 2,376 bp fragment of HIV-1 used to replace the deleted 4,981 bp and 472 bp SHIV_{KU-2} sequences (SEQ ID NO:8 and SEQ ID NO:9, respectively) is designated SEQ ID NO:10.

In addition to the above, a 411 bp DNA fragment is deleted from the 3′ LTR of SHIV_{KU-2} to yield the Δ4-SHIV_{KU-2} DNA construct (SEQ ID NO:7). This deleted 3′ LTR sequence is designated SEQ ID NO:11. In the Δ4-SHIV_{KU-2} DNA construct the deleted 3′LTR sequences are replaced with 481 bp DNA sequence of the SV40 polyadenylation signal sequence that is designated SEQ ID NO:12.

### EXAMPLE 5B

### In Vivo Efficacy of Both the V7 and Δ4-SHIV_{KU-2} DNA Vaccines

Example 3 of Ser. No. 10/941,164 entitled “DNA Vaccines and Methods of Use” filed on Sep. 15, 2004 describes the efficacy of the V7 Vaccine from Example 4 and Δ4-SHIV_{KU-2} Vaccine from Example 5B. As discussed therein, although the experiment described above in Example 4B indicated the efficacy of the V7 vaccine lacking the rt gene and 3′ LTR, it was not clear whether the Δ4-SHIV_{KU-2} would be efficacious as a vaccine. The uncertainty stems from the fact that the current vaccine Δ4-SHIV_{KU-2} contains four deletions (rt, int, vif, and the 3′ LTR), each deletion corresponding to a portion of the viral genome important in pathogenicity and infectivity of the virus. The deletions were made in order to render the virus non-pathogenic, non-infectious and safe for use, but it was unknown whether these four deletions, in addition to the fact that a DNA rather than a live virus was being used, would render the vaccine incapable of providing protection against HIV-1.

Surprisingly, the present virus proved to be just as efficient at inducing protection against heterologous SHIV 89.6P as the V7 Vaccine described in the live virus comparison of Example 4B.

Three macaques were injected intramuscularly with 5 mg of the V7 Vaccine while three other macaques were injected intramuscularly with 5 mg of the present Δ4-SHIV_{KU-2} DNA. The injections were repeated eleven weeks later, and the animals were challenged intravenously with undiluted stock of SHIV 89.6P six weeks after the second immunization. All six of the animals developed ELISPOT™ responses to the vaccine three weeks after the first injection, the responses declining approximately three weeks later to undetectable levels. The responses appeared once again only one week after the second injection, and again declined to low levels. Only minimal responses were detected at the time of challenge. By one week after challenge, each of the animals had developed high titers of viral replication, which were matched by a powerful CMI (cell-mediated immune) response. By two weeks post-challenge, the viral burdens in the animals declined to levels between ten- and twenty-fold less than concentrations observed one week earlier. None of the animals lost CD4 T-cells. The ability of the DNAs to induce protection after only two injections underscores the potency of the DNA vaccines, and the results of the experiment clearly showed that that, despite the additional deletions, the Δ4-SHIV_{KU-2} DNA construct DNA vaccine (SEQ ID NO:7) of the present invention was just as effective as the V7 vaccine, which in turn was just as effective as the live virus vaccine.

### EXAMPLE 6

### Efficacy of SV40 PolyA Tail substituted for 3′LTR

This example is described in Example 4 of parent patent application Ser. No. 10/941,164 entitled “HIV Vaccine and Method of Use.” More specifically, the rt gene, int gene, vif gene, and 3′ LTR was deleted in the passaged, highly pathogenic SHIV_{KU-2} virus were made to create a novel vaccine. A further experiment was performed to compare the utility of the SV40 polyadenylation sequences as substitutes for the 3′ LTR sequence.

This was accomplished by comparing the ability of the V5 embodiment ΔrtSHIV_{KU-2} DNA vaccine with an intact 3′LTR (SEQ ID NO:1) from Example 2 and the V6 embodiment Δrt Δ3′LTRSHIV_{KU-2} DNA vaccine having the 3′LTR substituted with an SV40 polyadenylation sequence (SEQ ID NO:3) from Example 3 to express vector-encoded viral proteins. Performance of the two DNA molecules was compared in transfected primary human fibroblasts, the human embryonic kidney epithelial cell line 293, and in Jurkat cells, for expression of viral proteins in intra and extracellular compartments. The DNAs were also compared for duration of expression and for the amount of protein production, as well as for posttranslational modification and cleavage of precursor proteins. It was determined that the 3′ LTR deleted V6 embodiment SHIV_{KU-2} DNA vaccine construct, surprisingly, was more efficient in producing viral proteins than the V5 embodiment SHIV_{KU-2} DNA vaccine construct having both LTRs. The duration of protein production was also longer in the 3′ LTR deleted vaccine. Immunoprecipitation analysis revealed that deletion of the 3′ LTR resulted in rapid cleavage of the gag precursor, yielding double the amount of p27 being exported to the extracellular compartment. Taken together, these data indicate that deletion of the 3′ LTR not only alleviates concerns about integration of the viral genome into host DNA, but also results in a more efficient expression of viral proteins.

### EXAMPLE 7

### Coadministration of Cytokines DNA Vaccines

It is contemplated that the vaccines of the present invention may be co-administered with one or more cytokines (either in protein form or DNA vectors) or the genes encoding for the cytokines may be incorporated into the DNA vector itself.

In this example, the coadministration of cytokines with Δ4-SHIV_{KU-2} DNA vaccine was investigated. A study was undertaken to ascertain whether the immune response induced by the present vaccine could be enhanced by co-administration of cytokines (for example, GM-CSF) DNA. BALB/C mice were immunized intramuscularly with a mixture of 100 μg of Δ4-SHIV_{KU-2} DNA and 25 μg of mouse GM-CSF DNA (Invitrogen). The injections were given twice, two weeks apart, and the mice were sacrificed one week after the second immunization. Splenocytes were tested for response to SIV Gag peptides divided into five groups in the ELISPOT™ assay. Even though the immunization doses were low and tissue samples were harvested early, before CMI responses could peak, all four animals that received GM-CSF DNA along with the vaccine DNA developed ELISPOT™ responses, varying from 20 to 40 cells/10⁶ splenocytes, whereas only 50% of the animals receiving the vaccine DNA alone developed such a response. The GM-CSF caused an impressive chemotactic effect, as evidenced by the large number of mononuclear cells that were concentrated at the site of injection. This effect attracted many more antigen-presenting dendritic cells to the site of the injection that evident in the animals that received the DNA vaccine only. Surprisingly, however, the mice that received both vaccine DNA and GM-CSF developed lower CMI titers that those receiving the DNA vaccine alone. That is, the number of viral protein specific ELISPOT™ positive cells generated by the vaccine alone was significantly higher than those generated by the vaccine plus GM-CSF. It is concluded that coadministration of the Δ4-SHIV_{KU-2} DNA vaccine with a cytokine such as GM-CSF may be desirable in instances where it is either prophylactically or therapeutically desirable to increase the number of injected subjects that develop activated splenocytes.

Thus, the present DNA vaccine is useful for providing protection against HIV. The DNA used in the present invention was derived from SHIV_{KU-2}, a virus that has a highly efficient replication strategy, making it highly pathogenic. The transcriptional machinery of the DNA was maintained by preserving the 5′ LTR that houses the promoter/enhancer sequences of the viral DNA. In addition, the 5′ LTR contains binding sites for transcription factors such as NFKB, NFAT, SP-1, and the like, and the binding site for the RNA of tat, a molecule unique to HIV and the lentivirus that is responsible for the transactivation of viral DNA. The integrase gene and the 3′ LTR were deleted to minimize the ability of the DNA to integrate into host cell DNA. Thus, the DNA cannot persist indefinitely in tissues. Furthermore, the deletion of the rt and vif genes crippled the ability of the genome to code for pathogenic, infectious virus. At the same time, the viral proteins encoded by the env, gag, vpu, tat, and nef genes were highly expressed in cells transfected with the DNA. The present DNA vaccine is highly immunogenic in macaques and elicits protective immunity against heterologous viruses. Importantly, the present vaccine can be used not only prophylactically, but also therapeutically in individuals already infected with HIV because the DNA may be injected at any time during a period when anti-retroviral drug therapy is in place.

The examples and disclosure provided above describe certain embodiments of the present invention, but are not meant to be limiting. It will be apparent to those of skill in the art, upon reading this disclosure, that the present invention may be modified in a number of ways without departing from the spirit or scope of the invention. For example, the env, gag, and nef genes described above could be excised and replaced with the corresponding genes from another subtype of HIV. Thus, the present vaccine could be used for immunization against various subtypes of HIV. Further, the env, gag, nef and other genes described above could be replaced with genes from other viruses, such as SARS and Hepatitis C. Thus, the present DNA vaccine, described above, could be used as an “engine” to drive expression of viral genes from other than HIV or SIV, thereby providing a DNA vaccine to a variety of other viruses. The present invention is limited only by the claims that follow.

What is claimed and desired to be secured by Letters Patent is as follows:

1. An immunogenic composition comprising a DNA molecule having a sequence encoding a plurality of viral proteins capable of stimulating an immune response against HIV, said DNA molecule generated by:
passaging a live SHIV virus to provide a virus isolate that is more pathogenic than said SHIV virus would be without said passaging, and then
rendering the combination of the plurality viral proteins non-pathogenic by disrupting the ability of the DNA molecule of said passaged virus to encode for at least one viral protein necessary for a pathogenic virus, said disrupting step including a deletion in the rt gene.

2. The composition of claim 1 wherein said passaged SHIV virus is rendered non-pathogenic by a deletion in the rt gene, int gene, and vif genes.

3. The immunogenic composition of claim 1 wherein said DNA molecule is generated by passaging said live SHIV virus with at least two successive passages in vivo through macaque bone marrow to render provide a virus isolate that is more pathogenic than said SHIV virus would be without said passaging.

4. The immunogenic composition of claim 1 wherein said SHIV virus is passaged such that said passaged virus infects a monkey and causes a monkey to develop AIDS-associated symptoms within about 32 weeks of infection.

5. The immunogenic composition of claim 1 wherein said SHIV virus is passaged such that 70% of inoculated subjects develop AIDS within six months after inoculation with the passaged virus.

6. The immunogenic composition of claim 1 wherein the sequence encoding the plurality of viral proteins capable of stimulating an immune response is selected from a group of coding sequences comprising the gag, pro, tat, rev, vpu, env, vpx, vpr and nef genes of either SIV or HIV.

7. The immunogenic composition of claim 1 comprising a DNA molecule having a sequence encoding a plurality of viral proteins capable of stimulating an immune response against HIV generated by:
at least two successive passages in vivo of a SHIV viral isolate through macaque bone marrow, said SHIV virus including a DNA sequence which includes a human HIV env protein, and wherein said passaged virus infects a monkey causing said monkey to develop AIDS-associated symptoms within about 32 weeks of infection; and
rendering the DNA non-pathogenic by disrupting the rt gene to render the rt gene non-functional.

8. The immunogenic composition of claim 1 further comprising disrupting an int gene and vif gene to render both genes non-functional.

9. The immunogenic composition of claim 1 wherein said DNA molecule is selected from the group consisting of SEQ ID NOs: 1, 3, and 5.

10. The immunogenic composition of claim 1 wherein said DNA molecule is selected from the group consisting of SEQ ID NO: 7.

11. A method of making a DNA immunogenic composition comprising:
serial passaging a live SHIV virus in macaques to increase the pathogenicity of said virus;
rendering the SHIV virus non-pathogenic by disrupting the ability of the DNA of said passaged SHIV virus to encode for at least one viral protein necessary for a pathogenic virus, said disrupting step including a deletion in the rt gene.

12. The method of claim 11 wherein said passaged SHIV virus is rendered non-pathogenic by a deletion in the rt gene, int gene, and vif genes from the live SHIV.

13. The method of claim 11 wherein said live SHIV virus undergoes at least two successive passages in vivo through macaque bone marrow.

14. The method of claim 11 wherein said live SHIV virus is passaged to render said virus more pathogenic than said virus would be without said passaging such that said passaged virus infects a monkey and causes a monkey to develop AIDS-associated symptoms within about 32 weeks of infection.

15. The method of claim 11 wherein said live SHIV virus is passaged to render said virus more pathogenic than said virus would be without said passaging such that said passaging such that 70% of inoculated subjects develop AIDS within six months after inoculation with the passaged virus.

16. The method of claim 11 wherein DNA immunogenic composition encodes a plurality of viral proteins capable of stimulating an immune response is selected from a group of coding sequences comprising the gag, pro, tat, rev, vpu, env, vpx, vpr and nef genes of either SIV or HIV.

17. The method of claim 11 wherein said step of serial passaging a live SHIV virus in macaques to increase the pathogenicity of said virus comprises at least two successive passages in vivo of a SHIV viral isolate through macaque bone marrow, and wherein said SHIV virus has a DNA sequence which includes a human HIV env protein, and wherein said passaged SHIV virus infects a monkey causing said monkey to develop AIDS-associated symptoms within about 32 weeks of infection.

18. The method of claim 17 further comprising disrupting an int gene and vif gene to render both genes non-functional.
